(19) United States
(12) Patent Application Publication
(10) Pub. No.: US 2014/0321601 A1
(43) Pub. Date: Oct. 30, 2014

(54) ACTIVE SHIELD FOR X-RAY COMPUTED TOMOGRAPHY MACHINE

(71) Applicant: Texas Instruments Incorporated, Dallas, TX (US)

(72) Inventor: Sreenivasan K. Koduri, Allen, TX (US)

(73) Assignee: Texas Instruments Incorporated, Dallas, TX (US)

(21) Appl. No.: 14/175,344

(22) Filed: Feb. 7, 2014

Related U.S. Application Data
(60) Provisional application No. 61/816,244, filed on Apr. 26, 2013.

(51) Int. Cl. A61B 6/03; G01N 23/04

(52) U.S. Cl. CPC A61B 6/032; G01N 23/046
USPC 378/4; 250/515.1

(57) ABSTRACT

An active shield for an X-ray computed tomography machine includes a radiation shielding substrate and a flexible circuit board wrapped around the substrate.

### PRIORITY

This application claims priority from U.S. provisional patent application Ser. No. 61/816,244 filed Apr. 26, 2013 for STRUCTURE AND METHOD FOR CONFIGURING SENSING ELEMENTS IN AN X-RAY COMPUTED TOMOGRAPHY (CT) of Sreenivasan K. Koduri, which is hereby incorporated by reference for all that it discloses.

### BRIEF DESCRIPTION OF THE DRAWINGS

FIG. 1 is a perspective view of a prior art X-ray computed tomography machine (“CT machine”).

FIG. 2 is schematic end elevation view of the gantry of a prior art CT machine.

FIG. 3 is a schematic side elevation view of a prior art X-ray sensor assembly of a CT machine.

FIG. 4 is a schematic isometric view of a scintillator of a prior art X-ray sensor assembly.

FIG. 5 is an isometric view showing the relationship between a prior art X-ray sensor assembly and signal processing electronics.

FIG. 6 is a schematic side elevation view of an active shield having an X-ray sensor assembly mounted on the first side thereof and signal processing electronics mounted on a second side thereof.

FIG. 7 is an isometric top view of an active shield.

FIG. 8 is an upside down cross-sectional view of active shield.

FIG. 9 is an isometric bottom view of an active shield and electronics mounted thereon.

FIG. 10 is a top plan view of one layer of a wraparound electrical circuit.

FIG. 11 is a partially cut away isometric view of another embodiment of an active shield.

FIG. 12 is schematic end elevation view of the gantry of a CT machine.

### DETAILED DESCRIPTION

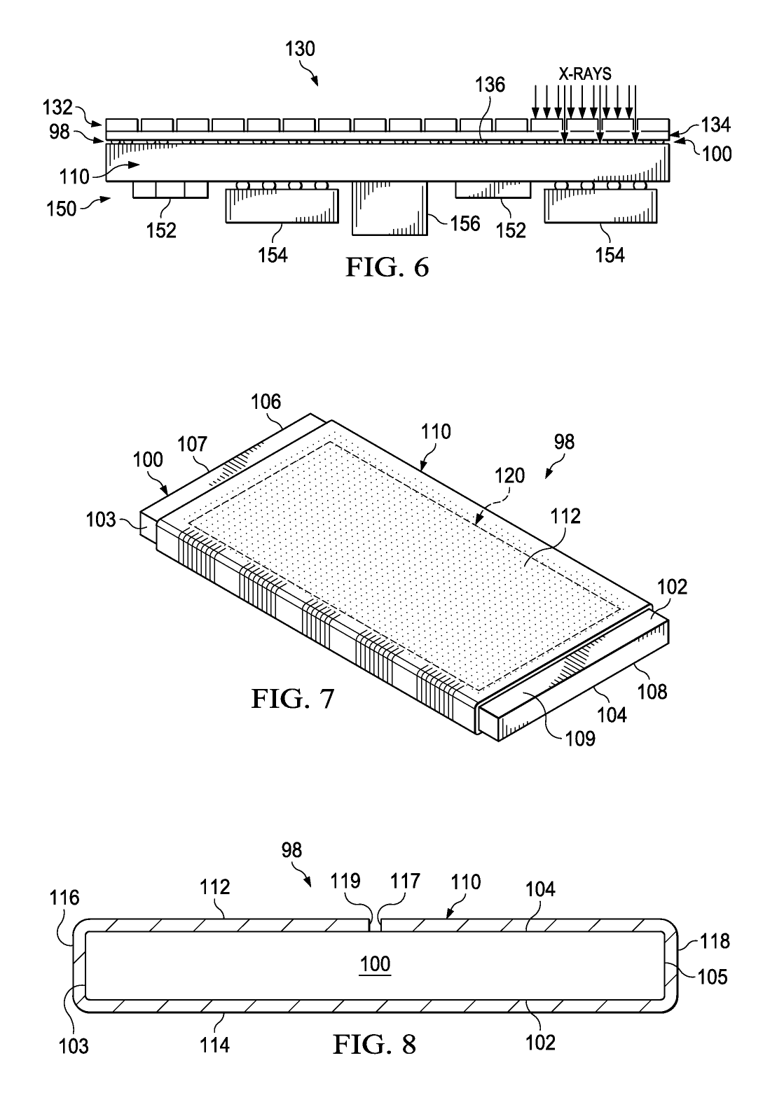

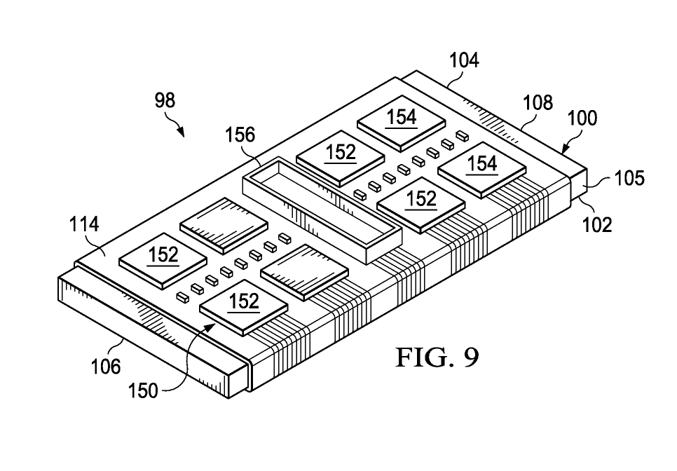

In general, an X-ray computed tomography machine 210 (“CT machine”) is described herein. As best shown in FIGS. 6-9 and 12, the CT machine 210 has a radiation shielding substrate 100 with a first side 102 and an opposite second side 104. It includes a wraparound electrical circuit 110 that is wrapped around the radiation shielding substrate 100. The CT machine also has an X-ray sensor assembly 130 that is adapted to generate sensor signals indicative of X-rays impinged on it. The sensor assembly 130 is connected to the wraparound electrical circuit 110 and is positioned adjacent to the first side 102 of the substrate. The CT machine also has a sensor signal processing assembly 150 which is connected to the wraparound electrical circuit 110. The signal processing assembly 150 is positioned adjacent to the second side 104 of the substrate 100. The signal processing assembly 150 is shielded from X-rays by the radiation shielding substrate 100.

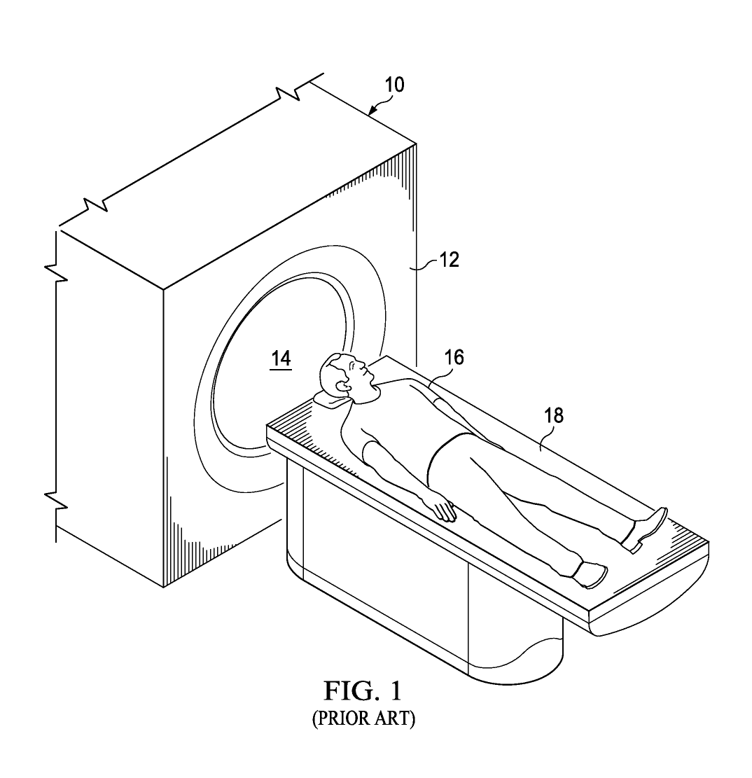

FIG. 1 illustrates a prior art X-ray computed tomography machine 10 (CT machine 10). The CT machine 10 has a CT housing 12 with a center opening 14 therein. During CT operation, a patient 16 lies on a horizontally disposed table 18. The table 18 is slowly moved through the central opening 14 as X-rays from the machine pass through the patient's body.

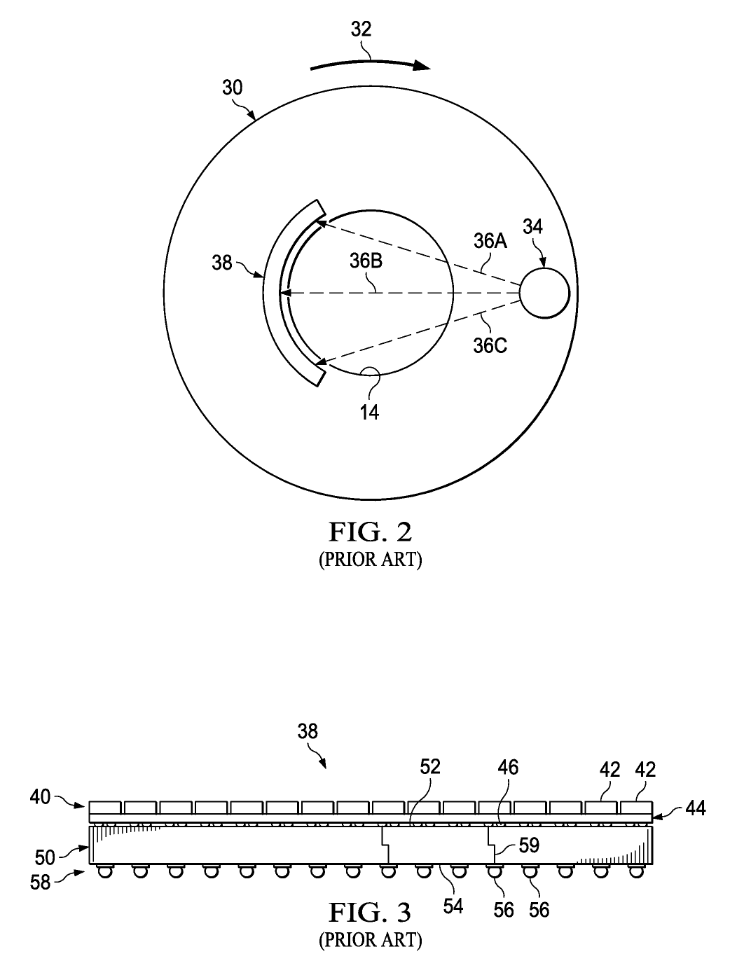

FIG. 2 is a schematic view of a gantry 30 of the prior art CT machine 10. The gantry 30 is a massive doughnut-shaped device that is positioned behind a front cover of the CT machine 10. The gantry 30 rotates in direction 32 as the patient is moved through the opening 14. The gantry 30 includes an X-ray tube 34 positioned on one side of the central hole 14. An X-ray sensor assembly 38 is positioned on the opposite side of the center opening 14. X-rays 36A, 36B, 36C from the X-ray tube 34 are sensed by the sensor assembly 38. Analog signals from the sensor assembly 38 are processed and used to create three-dimensional images of the interior of the patient's body.

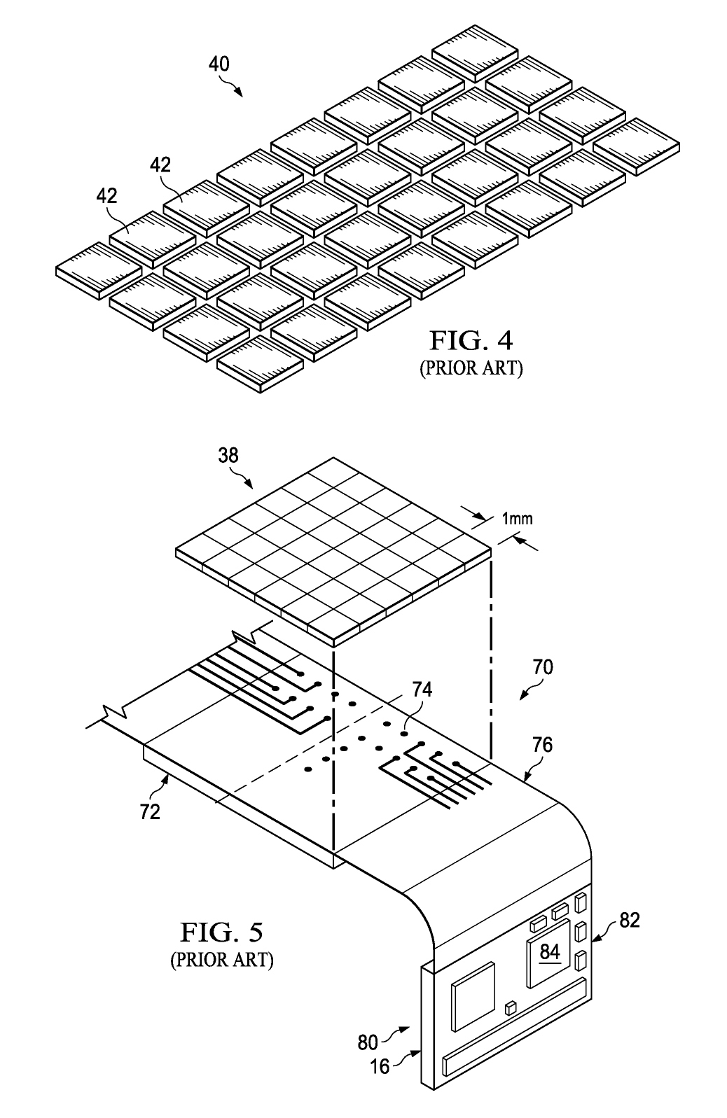

FIG. 3 is a schematic side elevation view of a portion of the prior art X-ray sensor assembly 38. Sensor assembly 38 includes a scintillator array 40 mounted on a photo detector array 44 that is, in turn, mounted on a ceramic substrate 50. FIG. 4 is an enlarged isometric view of a top portion the scintillator array 40, which comprises a grid of scintillator pixels 42. As shown by FIG. 3, the scintillator array 40 is supported on a photo detector array 44 having a plurality of pixels (not shown individually) corresponding to the pixels 42 of the scintillator array 40. (The term “pixel” as used herein may refer to an element of an image sensor as well as an element of an image.) The scintillator and photo detector pixels need to be held with high levels of geometric tolerances against mechanical, thermal, gravitational, and aging effects. Typically a thick ceramic substrate 50 is used to provide the necessary support. The ceramic substrate 50 has flat upper and lower surfaces 52, 54. A plurality of conductor pads (not shown) are provided on the top surface 52 of substrate 50 in alignment with conductors 46 on the photo detector assembly 44. Electrical contacts such as balls 56 of a ball grid array 58 are provided on the bottom surface 54 of the ceramic substrate 50. Conductors 59 extending through vias in the substrate 50 connect conductor pads (not shown) on the top surface 52 to corresponding ball conductors 56 on the bottom surface 54.

As illustrated by FIG. 5, the X-ray sensor assembly 38 is connected to signal processing electronics 80 which process analog signals from the photo detector array 44. The analog signals from the photo detector array can be corrupted if transmitted over long distances. To avoid such signal corruption the analog signals would ideally be filtered and digitized by electronics in close proximity to the photo detector 44. The digital signals thus produced could then be safely transmitted, stored, and further processed with no loss of integrity.

X-rays that strike the scintillator array 40 are absorbed by it. However some amount of radiation may escape from the sensor assembly 38 due to gap's between scintillator pixels 42, or because some X-rays pass entirely through the sensor assembly 38. Such stray X-rays can significantly alter the characteristics of signal processing electronics placed in the proximity of the sensor assembly 38.

Various techniques have evolved to prevent damage to the signal processing electronics. Such techniques generally involve use of additional printed circuit boards (PCB's) to route the signals to signal processing electronics located a substantial distance away from the sensor assembly. An example of such a prior art assembly is shown in FIG. 5. An analog signal transfer assembly 70 transfers analog signals from the X-ray sensor assembly 38 to a signal processing assembly 80. The analog signal transfer assembly 70 includes a first conventional PCB 72. The ball grid array 58 on the ceramic substrate 50 is placed in electrical contact with a corresponding conductor array 74 on the conventional PCB 72. A flexible PCB 76 connects the first conventional PCB 72 to a second conventional PCB 82 having analog signal processing electronics 84 mounted on it. A portion of the flexible PCB 76 and the second conventional PCB 82 extend generally perpendicular to the first conventional PCB 72 and parallel to the direction of X-rays from the X-ray tube 34. Although this structure places the signal processing electronics 82 at a distance from the source of the X-rays it has a number of drawbacks. Such structure undesirably increases the distance that the analog signals must travel to reach the signal processing electronics. Such structure also adds undesirable stack height (radial height) and mass to the entire X-ray sensor/signal processing assembly. Because of the rapid rotation of the gantry, significant centrifugal forces are generated that cause such structures to be mechanically unstable and electrically unreliable.

FIG. 6 illustrates an X-ray sensing and signal processing assembly that may be used to overcome the above described problem in CT machines. An X-ray sensor assembly 130 is mounted on one side of an active shield assembly 98. The X-ray sensor assembly 130 may comprise a scintillator array 132 and a photo detector array 134, which may be the same as the prior art scintillator array 40 and photo detector array 44 described above. Signal processing electronics 150 are positioned on the other side of the active shield assembly 98. The signal processing electronics 150 may comprise, for example, passive circuit devices 152 and integrated circuit packages 154. An electrical connector assembly 156 routes processed digital signals from the electronics 150 to other system electronics such as storage devices and displays (not shown).

Because the signal processing electronics 150 are positioned on the side of the active shield assembly 98 that is opposite the side facing the X-ray source, the electronics 150 are shielded from the X-rays. Also, because the electronics 150 are located close to the X-ray sensor assembly 130 the analog signals from the sensor assembly 130 travel only an advantageously short distance to the electronics 150. The analog signals are transmitted through a wraparound electric circuit 110, which is described below.

FIGS. 7-9 illustrate various features of the active shield assembly 98 shown in FIG. 6. The active shield assembly 98 comprises a radiation shielding substrate 100. The substrate 100 is constructed from a radiation blocking material such as tungsten or lead and has a first side 102 and opposite second side 104. The two sides 102 and 104 may comprise flat, generally parallel surfaces. The two sides 102, 104 are connected by smaller lateral sides 103, 105. The substrate 100 has a first longitudinal end 106 and a second longitudinal end 108. A typical thickness range for the substrate 100 may be about 0.5 mm to about 2.0 mm. Thus the X-ray sensor assembly 130 may be positioned less than about 3 mm from the signal processing electronics 150. An advantage of using a tungsten substrate 100, in addition to its excellent radiation shielding characteristics, is that it has a very low coefficient of thermal expansion. This helps in creating a very rigid, flat surface that does not change dimensions much with temperature. Thus a stable surface is provided for supporting the X-ray sensor assembly 130.

The active shield assembly 98 also comprises a wraparound electrical circuit 110, an embodiment of which is shown in FIGS. 7-10. The wraparound circuit 110 comprises a first larger portion 112 that engages and substantially covers the first side 102 of the substrate 100 and a second larger portion 114 that engages and substantially covers the second side 104 of the substrate 100. The wraparound circuit 110 also comprises third and fourth smaller portions 116,118 that engage and substantially cover the small lateral side portions 103, 105 of the substrate 100. Wrap around electrical circuit 110 has terminal end portions 117, 119, which are positioned adjacent one another when the circuit 110 is wrapped around the substrate 100, as that shown in FIG. 8.

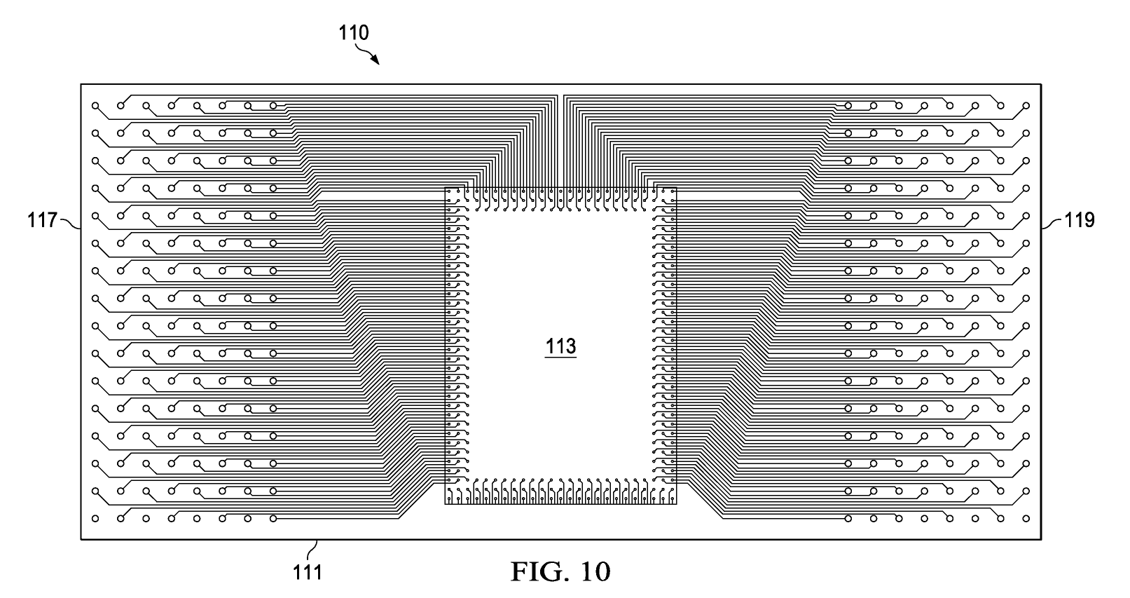

One layer of this flexible printed circuit board (“flex PCB”) type wraparound electrical circuit 110 is illustrated in FIG. 10. It includes a sheet of flexible nonconductive material 111, such as Kapton, polymer, etc., having a plurality of copper traces 113 formed on it. The traces 113 route analog signals from the photo detector assembly 134, which is located on one side 102 of the radiation blocking substrate 100, to the signal processing electronics 150 on the other side 104. In one embodiment each of the traces has a length no longer than about 100 mm. The void 115 in the center of the sheet 111 is the region where the signal processing electronics 150 would be located. The wrap around electrical circuit 110 may have one layer or more than one layer of the type illustrated in FIG. 10. In a multiple layer wraparound electrical circuit structure the layers would be interconnected by vias. One of these layers can be used as an electrical ground to isolate the high-speed signals from each other and from the radiation shielding substrate 100. It is also possible to electrically connect the ground of the wraparound electrical circuit 110 to the substrate 100 to eliminate any induced eddy currents or electrostatic build up.

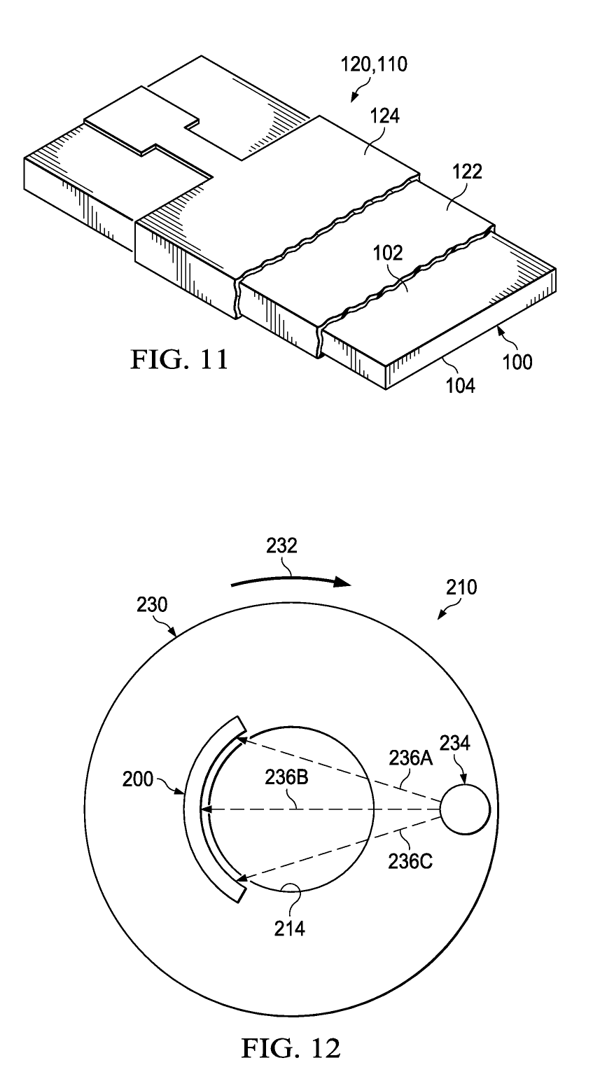

In another embodiment, as shown in FIG. 11, the wraparound electrical circuit 110 may be a metal clad structure 120. It includes a dielectric layer 122 that is adhered to the entire surface of the radiation shielding substrate 100. A patterned circuit layer 124 is provided on top of the dielectric layer 122, as by photolithography or other means.

As shown in FIG. 12, the active shield 98, X-ray sensor assembly 130 and sensor signal processing electronics 150 (referred to collectively as assembly 200) may be used in a CT machine 210. The CT machine 210 may have the same basic construction as the CT machine 10 described above with reference to FIGS. 1 and 2, except that assembly 200 replaces the X-ray sensor assembly 38 and the signal processing electronics associated therewith.

Multiple active shields 98 may be tiled together, to create a continuous larger active shield structure that supports a large X-ray sensor assembly 130 on one side and sensor signal processing electronics 150 on the other side. End portions of multiple substrates 100 could have features allowing them to be locked together, for example, dovetail joints. The wraparound electric circuit 110 maybe constructed to expose end portions 107, 109 of the substrate 100 to facilitate such locking connection.

It will be appreciated from the above disclosure that a method of generating digital signals representative of an image of a subject may include transmitting X-rays 236A, 236B, 236C through the subject from an X-ray source 234 on one side of the subject to a sensor assembly 130 supported on one side of an active shield 98 having opposite first and second sides, the active shield 98 being located on a side of the subject opposite to the side where the X-ray source 234 is located. The method may further include routing analog signals generated by the sensor assembly 130 to signal processing electronics 150 supported on the side of the active shield 98 opposite the side supporting the sensor assembly 130. The routing may comprise routing the analog signals through a wraparound circuit portion 110 of the active shield 98 that is wrapped around an X-ray blocking substrate portion 100 of the active shield 98.

Although certain specific embodiments of an active shield for a CT machine have been described in detail above, it will be understood by those skilled in the art after reading this disclosure that the active shield described herein could be variously otherwise embodied. For example, although the specific embodiment of a CT machine that is described herein is a medical CT machine, the CT machine features described herein are also applicable to other types of CT machines such as industrial CT machines used for imaging solder joints on printed circuit boards. The appended claims are intended to cover such alternative embodiments, except to the extent limited by the prior art.

What is claimed is:

1. An X-ray computed tomography machine (“CT machine”) comprising:
a radiation shielding substrate having a first side and an opposite second side;
a wraparound electrical circuit wrapped around said substrate;
an X-ray sensor assembly adapted to generate sensor signals indicative of X-rays impinged thereon, said sensor assembly being connected to said wraparound electrical circuit and positioned adjacent to said first side of said substrate; and
a sensor signal processing assembly connected to said wraparound electrical circuit and positioned adjacent to said second side of said substrate.

2. The CT machine of claim 1 wherein said wraparound electrical circuit comprises a flexible printed circuit board.

3. The CT machine of claim 1 wherein said wraparound electrical circuit comprises a dielectric layer formed on said radiation shielding substrate and a circuit layer formed on said dielectric layer.

4. The CT machine of claim 1 wherein said wraparound electrical circuit substantially covers said first side and said second side of said substrate.

5. The CT machine of claim 1 wherein said sensor assembly comprises:
a scintillator array that converts X-rays to light; and
a light detector array that receives light from said scintillator array and converts it to an analog electrical signal.

6. The CT machine of claim 4 wherein said wrap around electrical circuit transmits said analog electrical signals from said light sensor array to said sensor signal processing electronics and wherein said sensor signal processing electronics coverts said analog electrical signals into digital signals.

7. The CT machine of claim 1 wherein said wraparound electrical circuit comprises a plurality of circuit traces, wherein each of said plurality of circuit traces has a length no longer than about 100 mm.

8. The CT machine of claim 1 wherein said radiation shielding substrate is made from at least one of a metal and a metal alloy.

9. The CT machine of claim 1 wherein said radiation shielding substrate is made from tungsten.

10. The CT machine of claim 8 wherein the distance between the first and second surfaces of the substrate is between about 0.5 mm and 2 mm.

11. The CT machine of claim 1 wherein said sensor assembly is positioned less than about 3 mm from said sensor signal processing electronics.

12. The CT machine of claim 1 wherein said flexible circuit board comprises a plurality of circuit layers.

13. The CT machine of claim 12 wherein one of said plurality of circuit layers comprises a ground layer.

14. The CT machine of claim 13 wherein said ground layer is connected to said radiation shielding substrate.

15. An active shield for an X-ray computed tomography machine (“CT machine”) comprising:
a radiation shielding substrate; and
a wraparound electrical circuit wrapped around said substrate.

16. The active shield of claim 15 wherein said radiation shielding substrate has a first side and a second side and wherein said wraparound electrical circuit is adapted to route electrical signals from a sensor assembly positioned adjacent said first side of said substrate to signal processing electronics positioned adjacent said second side of said substrate.

17. The active shield of claim 16 wherein said substrate is adapted to mechanically support said sensor assembly and said signal processing electronics.

18. The active shield of claim 17 wherein said radiation shielding substrate is adapted to shield said signal processing electronics positioned adjacent said second side of said substrate from an X-ray source positioned on said first side of said substrate.

19. A method of generating digital signals representative of an image of a subject comprising:
transmitting X-rays through the subject from an X-ray source on one side of the subject to a sensor assembly supported on one side of an active shield having opposite first and second sides, the active shield being located on a side of the subject opposite to the side where the X-ray source is located; and
routing analog signals generated by the sensor assembly to signal processing electronics supported on the side of the active shield opposite the side supporting the sensor assembly.

20. The method of claim 19 wherein said routing comprises routing the analog signals through a wraparound circuit portion of the active shield that is wrapped around an X-ray blocking substrate portion of the active shield.
